(19) United States
(12) Patent Application Publication
(10) Pub. No.: US 2023/0233141 A1
(43) Pub. Date: Jul. 27, 2023

(54) ELECTRONIC DEVICE FOR PREDICTING AND DIAGNOSING SCOLIOSIS AND ITS OPERATING METHOD

(71) Applicant: Mi Kyoung JI, Jeju-si (KR)

(72) Inventor: Mi Kyoung JI, Jeju-si (KR)

(21) Appl. No.: 18/154,108

(22) Filed: Jan. 13, 2023

Related U.S. Application Data
(63) Continuation of application No. PCT/KR2021/008847, filed on Jul. 9, 2021.

(30) Foreign Application Priority Data
Jul. 13, 2020 (KR) 10-2020-0085995
Apr. 14, 2021 (KR) 10-2021-0048299

(51) Int. Cl. A61B 5/00; A61H 7/00

(52) U.S. Cl. CPC A61B 5/4561; A61H 7/007

(57) ABSTRACT

A method of predicting and diagnosing a diseases using an electronic device may include tracking an emotional or physiological change through a galvanic skin response; tracking a vertebral level or a peripheral nerve through spinal column scanning using a sensor unit; and automatically verifying the vertebral level associated with an emotional or physiological phenomenon and identifying a pain area through a combination of the galvanic skin response and spinal column scanning, and may predict and diagnose the disease through the identified pain area.

### CROSS-REFERENCE TO RELATED APPLICATIONS

This application is a bypass continuation application of International Patent Application No. PCT/KR2021/008847, filed on Jul. 9, 2021, which claims priority to Korean Applications No. 10-2020-0085995, filed Jul. 13, 2020, and No. 10-2021-0048299, filed Apr. 14, 2021, each of which is hereby incorporated by reference in its entirety.

### TECHNICAL FIELD

The following example embodiments relate to a method and apparatus for predicting and diagnosing a disease, and more particularly, to an electronic device for predicting and diagnosing a disease based on deep learning and an operating method thereof. Also, the following example embodiments relate to an electronic device for predicting and diagnosing scoliosis and an operating method thereof, and more particularly, to an electronic device for predicting and diagnosing scoliosis that may verify a degree of curvature of vertebral column through pressure or inclination of left and right sides of the vertebral column and an operating method thereof.

### BACKGROUND

Spine refers to a line of bones that support a main skeleton of a human including neck, back, waist, hip, and tail. To diagnose a spinal disease or a disease related to the spine, a picture of the spinal column is taken using X-ray, CT, or MRI and the disease is predicted and diagnosed according to an expert’s judgment from the picture of the spinal column. However, in the case of using X-ray, CT, or MRI, excessive cost may be used to acquire data every time and pictures need to be taken while continuously exposed to radiation for diagnosis and rehabilitation.

Scoliosis refers to one of representative spinal deformities. There is a curve in the human spine, which represents a curved state or a bent state. The curve of the spinal column includes a normal curve that appears in normal people and an abnormal curve that does not appear in normal people.

A different action needs to be taken according to a degree of scoliosis. However, in many cases, the disease is not recognized or only simple observation measures are taken, which frequently leads to degrading the disease.

### SUMMARY

Example embodiment describe a method and apparatus for predicting and diagnosing a disease, and more particularly, provide technology for automatically verifying a vertebral level associated with an emotional or physiological phenomenon and identifying a pain area through a combination of galvanic skin response and spinal column scanning based on deep learning.

Example embodiments also provide a disease prediction and diagnosis method and apparatus that may automatically verify a vertebral level associated with an emotional or physiological phenomenon and identify a pain area through a combination of galvanic skin response and spinal column scanning by continuously tracking the vertebral level while scanning vertebral column according to the vertebral level and by monitoring the galvanic skin response when the emotional or physiological phenomenon including pain appears at a level of a specific portion of the vertebral column.

Example embodiments also describe an electronic device for predicting and diagnosing scoliosis and an operating method thereof, and more particularly, technology for verifying a degree of curvature of vertebral column through pressure or inclination of left and right sides of the vertebral column.

Example embodiments also provide an electronic device for predicting and diagnosing scoliosis that may predict or diagnose scoliosis by measuring a degree of curvature of vertebral column through pressure or inclination of left and right sides of the vertebral column and may prevent the scoliosis by massaging a pain area or a portion with a relatively great difference in the pressure or the inclination and an operating method thereof.

An operating method of an electronic device according to an example embodiment may include tracking, by a galvanic skin response unit of the electronic device, an emotional or physiological change through galvanic skin response; tracking, by a spinal column scanning unit of the electronic device, a vertebral level or a peripheral nerve through spinal column scanning using a sensor unit; and automatically verifying, by a pain area identification unit of the electronic device, the vertebral level associated with an emotional or physiological phenomenon and identifying a pain area through a combination of the galvanic skin response and the spinal column scanning.

The tracking the vertebral level or the peripheral nerve through the spinal column scanning using the sensor unit may include measuring, by the spinal column scanning unit of the electronic device, a length of the vertebral column and tracking and verifying the vertebral level using at least one of an optical sensor, a pressure sensor, and an ultrasonic sensor.

The automatically verifying the vertebral level associated with the emotional or physiological phenomenon and the identifying the pain area through the combination of the galvanic skin response and the spinal column scanning may include monitoring, by the pain area identification unit of the electronic device, the galvanic skin response when the emotional or physiological phenomenon including pain appears at a level of a specific portion of the vertebral column and automatically verifying the vertebral level associated with the emotional or physiological phenomenon and identifying the pain area through the combination of the galvanic skin response and the spinal column scanning.

The method may further include tracking, by a neurological examination history taking unit of the electronic device, a disease and a symptom through neurological examination history taking prior to the galvanic skin response.

The method may further include inferring, by a nerve connector of the electronic device, a spinal nerve related to the emotional or physiological phenomenon through the identified pain area, verifying an organ controlled by the spinal nerve, connecting a related nerve to the organ, and tracking a symptom and a physiological change related to a disease state of the organ.

The method may further include collecting, by a data collector of the electronic device, data required between subjects through a result of the identified pain area and a result of tracking the symptom and the physiological change related to the disease state of the organ by connecting the related nerve to the organ; and predicting, by a deep learning unit of the electronic device, a current health state and a future health state of a subject through deep learning using the collected data.

The method may further include completing, by a disease prediction model modeling unit of the electronic device, a disease prediction model using a result of predicting the current health state and the future health state of the subject through deep learning.

The method may further include collecting, by a personal diagnostic result collector of the electronic device, a personal diagnostic result through a personal diagnostic device, and the current health state and the future health state of the subject may be predicted through deep learning using the result of the identified pain area, the result of tracking the symptom and the physiological change related to the disease state of the organ by connecting the related nerve to the organ, and the collected the personal diagnostic result.

A disease prediction and diagnosis apparatus according to another example embodiment may include a galvanic skin response unit configured to track an emotional or physiological change through galvanic skin response; a spinal column scanning unit configured to track a vertebral level or a peripheral nerve through spinal column scanning using a sensor unit; and a pain area identification unit configured to automatically verify the vertebral level associated with an emotional or physiological phenomenon and identify a pain area through a combination of the galvanic skin response and the spinal column scanning.

The disease prediction and diagnosis apparatus may further include an organ-and-related-nerve connector configured to infer a spinal nerve related to the emotional or physiological phenomenon through the identified pain area, to verify an organ controlled by the spinal nerve, to connect a related nerve to the organ, and to track a symptom and a physiological change related to a disease state of the organ.

The disease prediction and diagnosis apparatus may further include a data collector configured to collect data required between subjects through a result of the identified pain area and a result of tracking the symptom and the physiological change related to the disease state of the organ by connecting the related nerve to the organ; and a deep learning unit configured to predict a current health state and a future health state of a subject through deep learning using the collected data.

The disease prediction and diagnosis apparatus may further include a disease prediction model modeling unit configured to complete a disease prediction model using a result of predicting the current health state and the future health state of the subject through deep learning.

An operating method of an electronic device for predicting and diagnosing scoliosis according to an example embodiment may include measuring a degree of curvature of vertebral column through pressure or inclination of left and right sides of the vertebral column using a sensor; predicting pain likely to occur in the vertebral column through the degree of curvature of the vertebral column or comparing before and after and monitoring the degree of curvature of the vertebral column measured each time; and preventing scoliosis by massaging a pain area or a portion with a relatively great difference in the pressure or the inclination through an increase-and-decrease prediction and diagnosis of a curve that represents the degree of curvature of the vertebral column according to a monitoring result.

The method may further include collecting information on at least one of pain in the vertebral column, thoracic curve, neurological abnormal findings, and x-ray study prior to verifying the degree of curvature of the vertebral column. The pain may be predicted or the degree of curvature of the vertebral column may be monitored through the degree of curvature of the vertebral column that is measured based on the collected information.

The method may further include tracking an emotional or physiological change through a galvanic skin response. The scoliosis may be prevented by massaging according to the tracked emotional or physiological change.

The method may further include reselecting and operating a previous process through feedback after preventing the scoliosis by massaging the pain area or the portion with the relatively great difference in the pressure or the inclination. The reselecting and the operating the previous process through the feedback may include measuring the degree of curvature of the vertebral column again and comparing before and after and monitoring the degree of curvature of the vertebral column.

The measuring the degree of curvature of the vertebral column may include scanning the vertebral column using a device that moves along the vertebral column and measuring the degree of curvature of the vertebral column through the pressure or the inclination of the left and the right sides of the vertebral column using a sensor connected to the device.

The measuring the degree of curvature of the vertebral column may include scanning the vertebral column using a pushing rod that is pressed along the vertebral column and measuring the degree of curvature of the vertebral column through the pressure or the inclination of the left and right sides of the vertebral column using a sensor connected to the pushing rod.

An electronic device for predicting and diagnosing scoliosis according to another example embodiment may include a vertebral column scanning unit configured to measure a degree of curvature of vertebral column through pressure or inclination of left and right sides of the vertebral column using a sensor; a scoliosis prediction-and-diagnosis unit configured to predict pain likely to occur in the vertebral column through the degree of curvature of the vertebral column or to compare before and after and monitor the degree of curvature of the vertebral column measured each time; and a scoliosis preventer configured to prevent scoliosis by massaging a pain area or a portion with a relatively great difference in the pressure or the inclination through an increase-and-decrease prediction and diagnosis of a curve that represents the degree of curvature of the vertebral column according to a monitoring result.

The electronic device may further include an information collector configured to collect information on at least one of pain in the vertebral column, thoracic curve, neurological abnormal findings, and x-ray study. The scoliosis prediction-and-diagnosis unit may be configured to predict the pain or monitor the degree of curvature of the vertebral column through the degree of curvature of the vertebral column that is measured based on the collected information.

The electronic device may further include a galvanic skin response unit configured to track an emotional or physiological change through a galvanic skin response. The scoliosis preventer may be configured to prevent the scoliosis by massaging according to the tracked emotional or physiological change.

The electronic device may further include a feedback unit configured to reselect and operate a previous process through feedback after preventing the scoliosis by massaging the pain area or the portion with the relatively great difference in the pressure or the inclination. The feedback unit is configured to measure the degree of curvature of the vertebral column again and to compare before and after and monitor the degree of curvature of the vertebral column.

According to some example embodiments, there may be provided a disease prediction and diagnosis method and apparatus that may automatically verify a vertebral level associated with an emotional or physiological phenomenon and identify a pain area through a combination of galvanic skin response and spinal column scanning by continuously tracking the vertebral level while scanning vertebral column according to the vertebral level and by monitoring the galvanic skin response when the emotional or physiological phenomenon including pain appears at a level of a specific portion of the vertebral column.

Also, according to some example embodiments, there may be provided an electronic device for predicting and diagnosing scoliosis that may predict or diagnose scoliosis by measuring a degree of curvature of vertebral column through pressure or inclination of left and right sides of the vertebral column and may prevent the scoliosis by massaging a pain area or a portion with a relatively great difference in the pressure or the inclination, and an operating method thereof.

### BRIEF DESCRIPTION OF DRAWINGS

FIG. 1 illustrates an example of explaining a spinal column scanning device according to an example embodiment.

FIG. 2 is a diagram illustrating an example of explaining an operation of a spinal column scanning device according to an example embodiment.

FIG. 3 is a diagram illustrating an example of an electronic device according to an example embodiment.

FIG. 4 is a diagram illustrating an example of a disease prediction and diagnosis apparatus according to an example embodiment.

FIG. 5 illustrates an example of explaining an operation of a disease prediction and diagnosis apparatus according to an example embodiment.

FIG. 6 is a flowchart illustrating an example of a disease prediction and diagnosis method according to an example embodiment.

FIG. 7 illustrates an example of measuring pressure and inclination using a thoracic cross-section and a sensor according to an example embodiment.

FIG. 8 illustrates an example of a vertebral column scanning device according to an example embodiment.

FIG. 9 illustrates an example of an operation of a vertebral column scanning device according to an example embodiment.

FIG. 10 illustrates another example of a vertebral column scanning device according to an example embodiment.

FIG. 11 is a diagram illustrating an example of a configuration of a vertebral column scanning device according to an example embodiment.

FIG. 12 is a diagram illustrating an example of an electronic device according to an example embodiment.

FIG. 13 is a diagram illustrating an example of an electronic device for predicting and diagnosing scoliosis according to an example embodiment.

FIG. 14 illustrates an example of an operating method of an electronic device for predicting and diagnosing scoliosis according to an example embodiment.

### DETAILED DESCRIPTION

Hereinafter, example embodiments will be described with reference to the accompanying drawings. However, various modifications may be made to the example embodiments and the scope of the disclosure should not be construed as being limited to the example embodiments. Also, the example embodiments are provided to more fully explain the disclosure to those skilled in the art. Shapes and sizes of components in the drawings may be exaggerated for clarity of description.

FIG. 1 illustrates an example of explaining a spinal column scanning device according to an example embodiment.

Referring to FIG. 1, a spinal column scanning device 100 according to an example embodiment may include a roller 110, a sensor unit 120, and a guide rail 130.

The roller 110 enables spinal column scanning by moving along a spinal column in contact with a body portion of a user. The roller 110 may be in at least one spherical shape or cylindrical shape. For example, the roller 110 may be in a shape similar to that of a dumbbell, but the shape of the roller 110 is not limited thereto.

The sensor unit 120 may verify a spinal level when the roller 110 moves along the spinal column. For example, the sensor unit 120 may include a pressure sensor, an ultrasonic sensor, an optical sensor, and the like, and may verify a spinal level as the roller 110 moves along the spinal column. Here, the sensor unit 120 may be provided below the roller 110 and may move with the roller 110, but a location of the sensor unit 120 is not limited thereto.

The guide rail 130 may guide the roller 110 to move from one direction to another direction. That is, as the roller 110 moves from one side to another side along the guide rail 130, the spinal column of the user may be scanned.

As described above, for example, the user may lie down on the top of the guide rail 130 or a plate or a bed to which the guide rail 130 is provided. Here, as the roller 110 moves along the guide rail 130, the user may verify a spinal level by scanning the spinal column through the sensor unit 120. Here, although a method of scanning the spinal column of the user while the user is lying down is described as an example, the spinal column may also be scanned while the user is in an upright state.

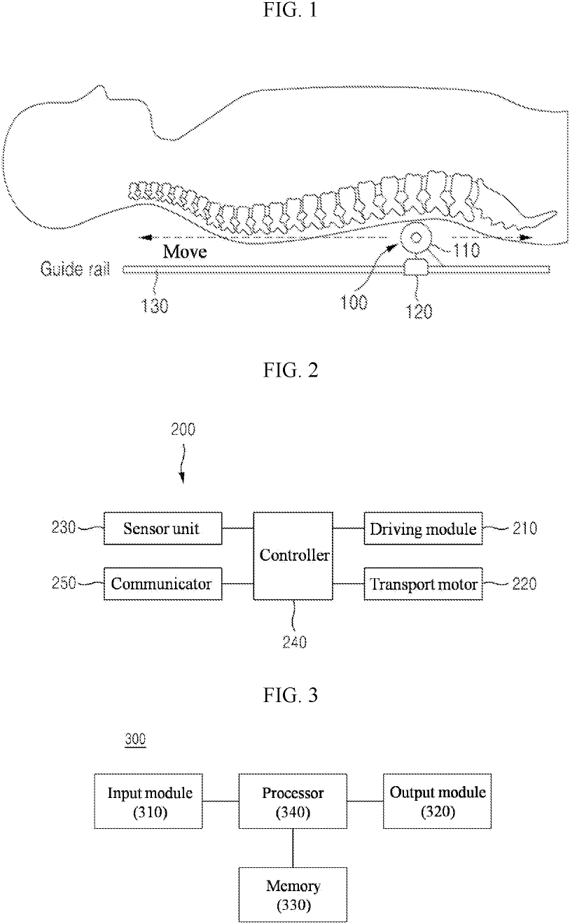

FIG. 2 is a diagram illustrating an example of explaining an operation of a spinal column scanning device according to an example embodiment.

Referring to FIG. 2, a spinal column scanning device 200 according to an example embodiment may include a driving module 210, a transport motor 220, a sensor unit 230, and a controller 240, and depending on example embodiments, may further include a communicator 250.

The driving module 210 may move along a spinal column in such a manner that a roller rotates and is in contact with a body portion of the user. The driving module 210 may move from one side to another side by way of the transport motor 220. Also, depending on example embodiments, the driving module 210 may adjust a height of a portion that makes a contact with the body according to preset strength.

The transport motor 220 may move the driving module 210 from one side to another side. Here, as the driving module 210 is moved from one side to the other side within a guide rail, the driving module 210 may move along the spinal column of the user.

The sensor unit 230 may include a pressure sensor, an ultrasonic sensor, an optical sensor, etc., and may verify a spinal level as the driving module 210 moves along the spinal column.

The controller 240 may operate and control the driving module 210, the transport motor 220, and the sensor unit 230, and may collect sensing data acquired from the sensor unit 230 or may transfer the sensing data to an external terminal through the communicator 250.

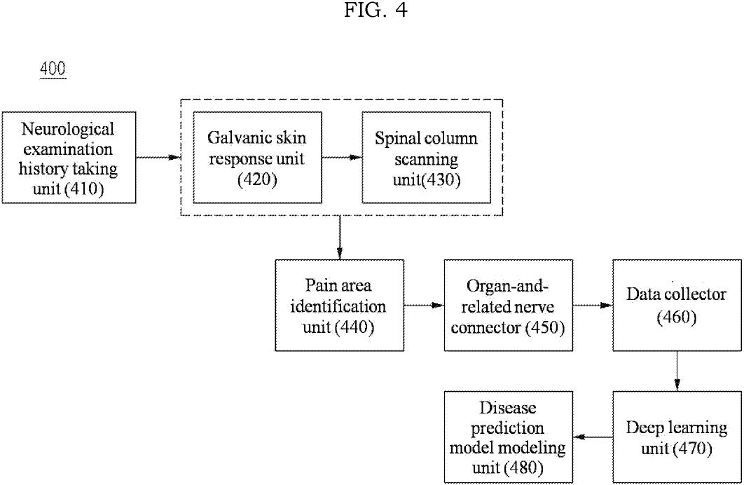

FIG. 3 is a diagram illustrating an example of an electronic device according to an example embodiment.

Referring to FIG. 3, an electronic device 300 according to an example embodiment may include at least one of an input module 310, an output module 320, a memory 330, and a processor 340.

The input module 310 may receive an instruction or data to be used for a component of the electronic device 300 from an outside of the electronic device 300. The input module 310 may include at least one of an input device configured for a user to directly input an instruction or data to the electronic device 300 and a communication device configured to receive an instruction or data through communication with an external electronic device in a wired or wireless manner. For example, the input device may include at least one of a microphone, a mouse, a keyboard, and a camera. For example, the communication device may include at least one of a wired communication device and a wireless communication device and the wireless communication device may include at least one of a near field communication device and a far field communication device.

The output module 320 may provide information to the outside of the electronic device 300. The output module 320 may include at least one of an audio output device configured to auditorily output information, a display device configured to visually output information, and a communication device configured to transmit information through communication with the external electronic device in a wired or wireless manner. For example, the communication device may include at least one of a wired communication device and a wireless communication device and the wireless communication device may include at least one of a near field communication device and a far field communication device.

The memory 330 may store data used by a component of the electronic device 300. Data may include input data or output data related to a program or an instruction related thereto. For example, the memory 330 may include at least one of a volatile memory and a nonvolatile memory.

The processor 340 may control a component of the electronic device 300 and may perform data processing or operation by executing the program of the memory 330. Here, the processor 340 may include a galvanic skin response unit, a spinal column scanning unit, and a pain area identification unit, a neurological examination history taking unit, an organ-and-related nerve connector, a data collector, a deep learning unit, and a disease prediction model modeling unit.

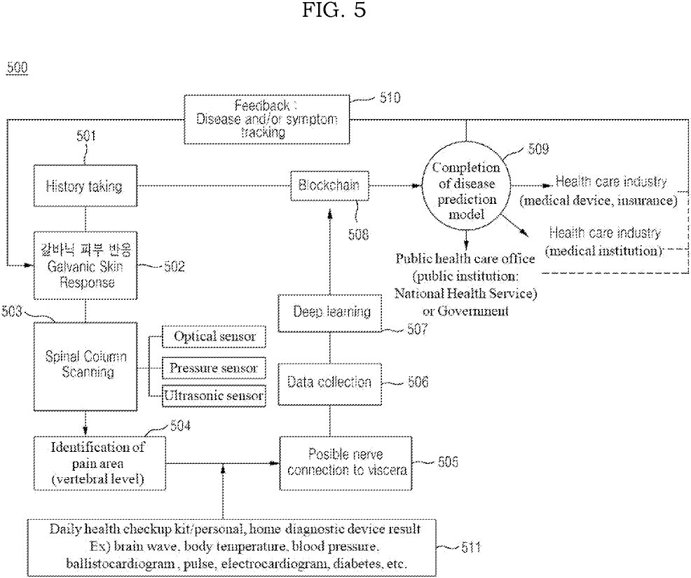

FIG. 4 is a diagram illustrating an example of a disease prediction and diagnosis apparatus according to an example embodiment.

Referring to FIG. 4, a disease prediction and diagnosis apparatus 400 according to an example embodiment may include a galvanic skin response unit 420, a spinal column scanning unit 430, and a pain area identification unit 440. Depending on example embodiments, the disease prediction and diagnosis apparatus 400 may further include a neurological examination history taking unit 410, an organ-and-related nerve connector 450, a data collector 460, a deep learning unit 470, and a disease prediction model modeling unit 480. Here, the disease prediction and diagnosis apparatus 400 may be included in the processor 340 of FIG. 3.

Initially, the neurological examination history taking unit 410 refers to a general medical examination process and through this process, may take notes, such as a birth date and a gender of a subject, a major symptom disease, and OPQRST. Information according to neurological examination history taking may be received by inputting information of the subject to the input device through a manager, such as a doctor, or by receiving input of the information from the subject.

The galvanic skin response unit 420 may track an emotional or physiological change through galvanic skin response.

Also, the spinal column scanning unit 430 may track a vertebral level or a peripheral nerve through spinal column scanning using a sensor unit.

The pain area identification unit 440 may automatically verify a vertebral level associated with an emotional or physiological phenomenon and may identify a pain area through a combination of the galvanic skin response and the spinal column scanning.

The organ-and-related nerve connector 450 may infer a spinal nerve related to the emotional or physiological phenomenon through the identified pain area, may verify an organ controlled by the spinal nerve, may connect a related nerve to the organ, and may track a symptom and a physiological change related to a disease state of the organ.

The data collector 460 may collect data required between subjects through a result of the identified pain area and a result of tracking the symptom and the physiological change related to the disease state of the organ by connecting the related nerve to the organ.

The deep learning unit 470 may predict a current health state and a future health state of a subject through deep learning using the collected data.

The disease prediction model modeling unit 480 may complete a disease prediction model using a result of predicting the current health state and the future health state of the subject through deep learning. The disease prediction model modeling unit 480 may predict and diagnose a disease using the disease prediction model and may use the same as new data.

Hereinafter, an operation and a configuration of a disease prediction and diagnosis apparatus will be described.

FIG. 5 illustrates an example of explaining an operation of a disease prediction and diagnosis apparatus according to an example embodiment.

Referring to FIG. 5, an operation of a disease prediction and diagnosis apparatus 500 included in an electronic device is illustrated. Neurological examination history taking (501) refers to a general medical examination process and through this process, may take notes, such as a birth date and a gender of a subject, a major symptom disease, and OPQRST. Through such neurological examination history taking, a disease and a symptom may be tracked.

A sensor for galvanic skin response (GSR) 502 may track emotion and stress by pain. Skin electrical conduction that is not under conscious control through galvanic skin resistance or galvanic skin potential may vary according to development of sympathetic nerve activity when an external stimulus is applied. Therefore, an emotional or physiological change may be tracked and observed.

For example, a galvanic skin response (GSR) sensor may convert a minute change in skin resistance and conductance to a measurable voltage using an internal high differential impedance operational amplifier. This voltage may be sampled by a controller of a sensor. Once a stimulus is detected, a sympathetic nervous system responds and many physiological changes occur, such as sweating in the sweat glands. This small change in skin moisture may change the skin and tissue conductivity measured by the sensor.

A vertebral level and a peripheral nerve may be tracked through spinal column scanning 503. For example, the spinal column may be scanned using an optical sensor, a pressure sensor, an ultrasonic sensor, and the like.

The spinal column scanning 503 may measure a length of the vertebral column and may track and verify a vertebral level using an optical sensor, a pressure sensor, an ultrasonic sensor, and the like. Also, since the vertebral level may be tracked through the spinal column scanning 503, a related adjacent spinal nerve may also be tracked. Here, the vertebral column refers to a state in which vertebrae (spinal column) and intervertebral disks (intervertebral cartilage, disc) are gathered to form a column as a longitudinal axis of the body.

Through this, a pain area may be identified (504). While scanning the vertebral column using technology for the aforementioned spinal column scanning 503 according to the vertebral level, the vertebral level may be continuously tracked. When an emotional (psychological) or physiological phenomenon including pain appears at a level of a specific portion of the vertebral column, it may be monitored with technology for the aforementioned galvanic skin response 502. Therefore, the vertebral level associated with the emotional (psychological) or physiological phenomenon as well as pain may be automatically verified through a combination of technology for the galvanic skin response 502 and the spinal column scanning 503.

By connecting a related nerve to an organ (possible nerve connection to viscera 505), a symptom and a physiological change related to a disease state of the organ may be tracked and observed. Based on an identified pain area result, a spinal nerve that may be involved with effect over human body (e.g., emotionally (psychologically) or physiologically) may be inferred. Also, it is possible to set a goal capable of tracking and observing a symptom and a physiological change related to a disease state of a corresponding organ by verifying a major organ controlled by the spinal nerve.

Necessary data may be collected (data collection 506). Clinically necessary data between subjects may be accumulated through technology related to neurological examination history taking (501), identification of the pain area (504), and connection of the related nerve to the organ (505).

Here, technology for deep learning 507 may be applied. The deep learning 507 may be enabled through the technology related to neurological examination history taking (501), identification of the pain area (504), and connection of the related nerve to the organ (505). Through this, a current health state of a subject and a future health state of the subject may be predicted.

Corresponding data may prevent abnormal data from being damaged or deformed through technology for blockchain 508.

Therefore, a disease prediction model 509 may be completed.

A result of the disease prediction model 509 may be fed back (510) and used as overlapping or new data. For example, information on a disease or a symptom may be delivered through the disease prediction model 509 and the disease may be easily predicted or diagnosed through galvanic skin response of the user. Also, as a degree of completion of the disease prediction model 509 increases, the disease prediction model 509 may be employed in the fields of health care industry and public health care.

In addition, the corresponding technology may be used as a disease prediction variable to increase reliability of a result value of the deep learning 507 using a home personal diagnostic device result 511, such as a heart rate, a ballistocardiogram, a brain wave, a blood pressure, an electrocardiogram, a blood sugar test kit, etc., between technology related to identification of the pain area (504) and technology related to connection of the possible nerve to the organ (505).

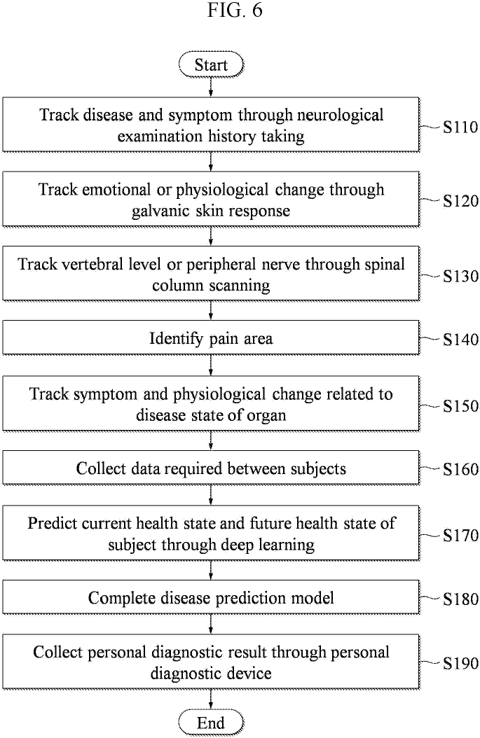

FIG. 6 is a flowchart illustrating an example of a disease prediction and diagnosis method according to an example embodiment.

Referring to FIG. 6, a disease prediction and diagnosis method using an electronic device according to an example embodiment may include operation S120 of tracking an emotional or physiological change through galvanic skin response, operation S130 of tracking a vertebral level or a peripheral nerve through spinal column scanning using a sensor unit, and operation S140 of automatically verifying the vertebral level associated with an emotional or physiological phenomenon and identifying a pain area through a combination of the galvanic skin response and the spinal column scanning, and may predict and diagnose a disease through the identified pain area.

Depending on example embodiment, the method may further include operation S110 of tracking a disease and a symptom through neurological examination history taking prior to the galvanic skin response.

Also, the method may further include operation S150 of inferring a spinal nerve related to the emotional or physiological phenomenon through the identified pain area, verifying an organ controlled by the spinal nerve, connecting a related nerve to the organ, and tracking a symptom and a physiological change related to a disease state of the organ.

Also, the method may further include operation S160 of collecting data required between subjects through a result of the identified pain area and a result of tracking the symptom and the physiological change related to the disease state of the organ by connecting the related nerve to the organ; and operation S170 of predicting a current health state and a future health state of a subject through deep learning using the collected data.

Also, the method may further include operation S180 of completing a disease prediction model using a result of predicting the current health state and the future health state of the subject through deep learning.

Also, the method may further include operation S190 of collecting a personal diagnostic result through a personal diagnostic device.

Hereinafter, each operation of the disease prediction and diagnosis method according to an example embodiment is further described.

The disease prediction and diagnosis method according to an example embodiment will be further described with reference to a disease prediction and diagnosis apparatus included in an electronic device of FIG. 4. The disease prediction and diagnosis apparatus 400 according to an example embodiment may include the galvanic skin response unit 420, the spinal column scanning unit 430, and the pain area identification unit 440. Depending on example embodiments, the disease prediction and diagnosis apparatus 400 may further include the neurological examination history taking unit 410, the organ-and-related nerve connector 450, the data collector 460, the deep learning unit 470, and the disease prediction model modeling unit 480.

In operation S110, the neurological examination history taking unit 410 may track a disease and a symptom through neurological examination history taking prior to the galvanic skin response. This refers to a general medical examination process and through this process, may take notes, such as a birth date and a gender of a subject, a major symptom disease, and OPQRST. Here, the neurological examination history taking unit 410 may receive Information according to neurological examination history taking by inputting information of the subject to the input device through a manager, such as a doctor, or by receiving input of the information from the subject.

In operation S120, the galvanic skin response unit 420 may track an emotional or physiological change through galvanic skin response. The galvanic skin response unit 420 may track emotion and stress by pain and may track the emotional or physiological change by verifying the galvanic skin response when performing spinal column scanning of the spinal column scanning unit 430.

In operation S130, the spinal column scanning unit 430 may track a vertebral level or a peripheral nerve through spinal column scanning using a sensor unit. Here, the spinal column scanning unit 430 may measure a length of the vertebral column and may track and verify the vertebral level using at least one of an optical sensor, a pressure sensor, and an ultrasonic sensor.

For example, a precise all-purpose ultrasonic sensor may be used as the ultrasonic sensor. The all-purpose ultrasonic sensor may precisely sense all from a location detection interval measurement to a solid powder or a liquid medium. This all-purpose ultrasonic sensor may measure an injection level height or deflection and may count the number of subjects, and may perform monitoring using a non-contact method. Regardless of a color or a surface material, the sensor may be used for a work without restrictions on time and occasion, and may be available even for a transparent or reflective object and may have no fog, dust, or contamination issues.

In operation S140, the pain area identification unit 440 may automatically verify a vertebral level associated with an emotional or physiological phenomenon and may identify a pain area through a combination of the galvanic skin response and the spinal column scanning.

In particular, the pain area identification unit 440 may automatically verify a vertebral level associated with an emotional or physiological phenomenon and identify a pain area through a combination of galvanic skin response and spinal column scanning by continuously tracking the vertebral level while scanning the vertebral column according to the vertebral level and by monitoring the galvanic skin response when the emotional or physiological phenomenon including pain appears at a level of a specific portion of the vertebral column.

In operation S150, the organ-and-related nerve connector 450 may infer a spinal nerve related to the emotional or physiological phenomenon through the identified pain area, may verify an organ controlled by the spinal nerve, may connect a related nerve to the organ, and may track a symptom and a physiological change related to a disease state of the organ.

In operation S160, the data collector 460 may collect data required between subjects through a result of the identified pain area and a result of tracking the symptom and the physiological change related to the disease state of the organ by connecting the related nerve to the organ.

In operation S170, the deep learning unit 470 may predict a current health state and a future health state of a subject through deep learning using the collected data. For example, the deep learning unit 470 may receive galvanic skin response information and the vertebral level and may predict and diagnose the disease through deep learning, and may also verify whether the vertebral level matches the existing information received from the neurological examination history taking unit 410. When such data is collected and another vertebral level is input, the deep learning unit 470 may easily predict and diagnose the disease.

In operation S180, the disease prediction model modeling unit 480 may complete a disease prediction model using a result of predicting the current health state and the future health state of the subject through deep learning. The disease prediction model modeling unit 480 may predict and diagnose a disease using the disease prediction model and may use the same as new data.

In operation S190, a personal diagnostic result collector may collect a personal diagnostic result through a personal diagnostic device. Therefore, reliability of a result value of deep learning may be increased by predicting the current health state and the future health state of the subject through deep learning using a result of the identified pain area, a result of tracking the symptom and the physiological change related to the disease state of the organ by connecting the related nerve to the organ, and the collected personal diagnostic result.

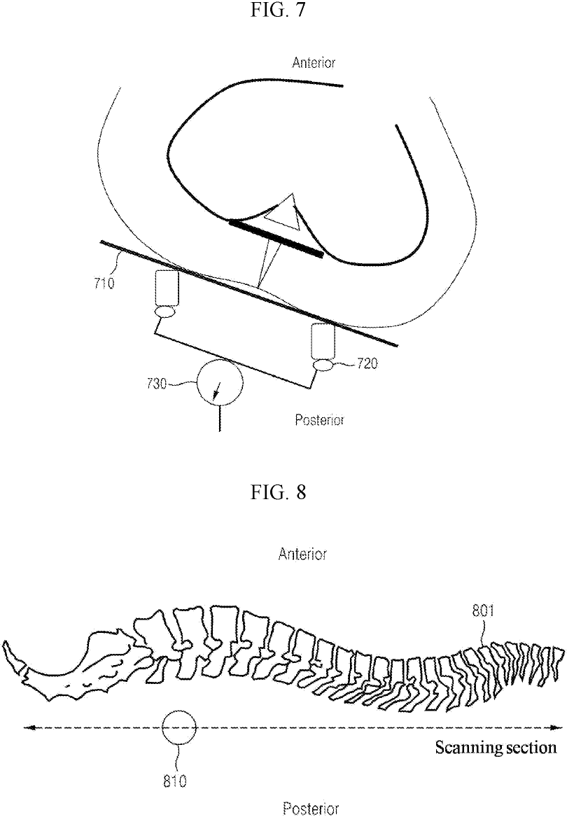

FIG. 7 illustrates an example of measuring pressure and inclination using a thoracic cross-section and a sensor according to an example embodiment.

Referring to FIG. 7, an operating method of an electronic device for predicting and diagnosing scoliosis according to an example embodiment may verify a degree of curvature of the vertebral column by measuring pressure or inclination 710 of left and right sides of the vertebral column using a sensor.

To this end, the scoliosis may be predicted or diagnosed by installing a pressure sensor 720 and/or an inclination sensor 730 to touch or connect to the thoracic and then measuring the degree of curvature of the vertebral column through pressure or inclination of left and right sides of the vertebral column. Also, the scoliosis may be prevented by intensively massaging a pain area or a portion with a relatively great difference in the pressure or the inclination.

Hereinafter, an operating method of an electronic device for predicting and diagnosing scoliosis according to an example embodiment is further described.

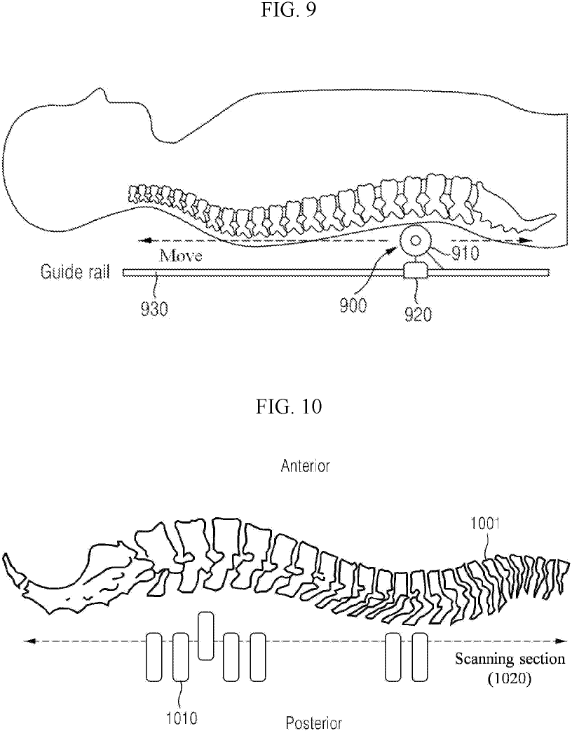

FIG. 8 illustrates an example of a vertebral column scanning device according to an example embodiment, and FIG. 9 illustrates an example of an operation of a vertebral column scanning device according to an example embodiment.

Referring to FIGS. 8 and 9, a vertebral column 801 may be scanned using a device 910 that exists at a specific position 810 in a scanning section of the vertebral column 801 and moves along the scanning section of the vertebral column 801, and the degree of curvature of the vertebral column 801 may be measured through pressure or inclination of left and the right sides of the vertebral column 801 using a sensor 920 connected to the device 910.

For example, a vertebral column scanning device 900 according to an example embodiment may include the device 910, the sensor 920, and a guide rail 930.

The device 910 enables vertebral column scanning by moving along the vertebral column 801 in contact with a body portion of a user. The device 910 may be formed in at least one spherical shape or cylindrical shape, and, for example, may be in a shape similar to that of a dumbbell, but the shape of the device 910 is not limited thereto.

When the device 910 moves along the vertebral column 801, the sensor 920 may verify the vertebral level. For example, the sensor 920 may include a pressure sensor, an inclination sensor, and the like, and may verify the vertebral level when the device 910 moves along the vertebral column. Also, the vertebral level may be verified using an optical sensor, an acceleration sensor, an angular sensor, etc., as well as the pressure sensor and the inclination sensor. Here, the sensor 920 may be provided below the device 910 and may move with the device 910, but a location of the sensor 920 is not limited thereto.

The guide rail 930 may guide the device 910 to move from one direction to another direction. That is, as the device 910 moves from one side to another side along the guide rail 930, the vertebral column 801 of the user may be scanned.

As described above, for example, the user may lie down on the top of the guide rail 930 or a plate or a bed to which the guide rail 930 is provided. Here, as the device 910 moves along the guide rail 930, the user may verify a vertebral level by scanning the vertebral column 801 through the sensor 920 that moves along together. Here, although a method of scanning the vertebral column 801 of the user while the user is lying down is described as an example, the vertebral column 801 may also be scanned while the user is in an upright state.

FIG. 10 illustrates another example of a vertebral column scanning device according to an example embodiment.

Referring to FIG. 10, vertebral column 1001 may be scanned using a pushing rod 1010 that is pressed along the scanning section 1020 of the vertebral column 1001 and a degree of curvature of the vertebral column 1001 may be measured through pressure or inclination of left and right sides of the vertebral column 1001 using a sensor connected to the pushing rod 1010. Here, the pushing rod 1010 may be pressed at a different level of pressing by the vertebral column 1001 in a state in which the user is lying down or standing upright and, here, may measure the degree of curvature of the vertebral column 1001 through the sensor.

FIG. 11 is a diagram illustrating an example of a configuration of a vertebral column scanning device according to an example embodiment.

Referring to FIG. 11, a vertebral column scanning device 1100 according to an example embodiment may include a driving module 1110, a transport motor 1120, a sensor 1130, and a controller 1140, and may further include a communicator 1150 depending on example embodiments.

The driving module 1110 may move along the vertebral column in such a manner that a device rotates and is in contact with a body portion of a user. The driving module 1110 may move from one side to another side by way of the transport motor 1120. Also, depending on example embodiments, the driving module 1110 may adjust a height of a portion that makes a contact with the body according to preset strength. Meanwhile, the driving module 1110 may be pressed at a different level of pressing by the vertebral column while the user is lying down or standing upright on the pushing rod.

The transport motor 1120 may move from one side to another side using the driving module 1110. Here, by moving the driving module 111 from one side to the other side within a guide rail, the transport motor 1120 may move along the vertebral column of the user. Meanwhile, in the case of using the pushing rod, the transport motor 1120 may be omitted.

The sensor 1130 may include a pressure sensor, an inclination sensor, and the like, and may verify a vertebral level when the driving module 1110 moves along the vertebral column.

The controller 1140 may operate and control the driving module 1110, the transport motor 1120, and the sensor 1130, and may collect sensing data acquired from the sensor 1130 or may transmit the same to an external terminal through the communicator 1150.

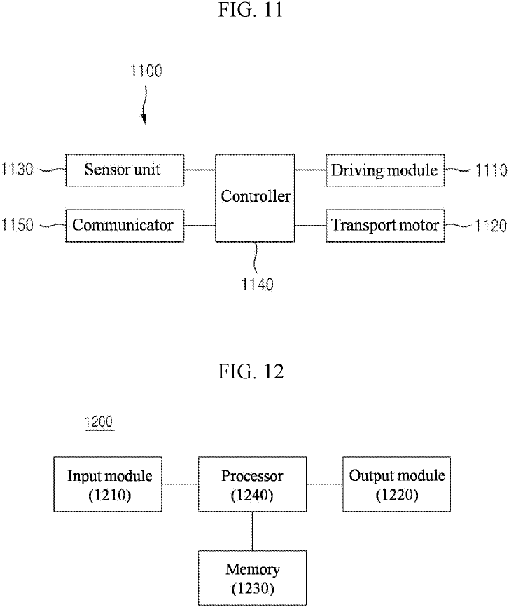

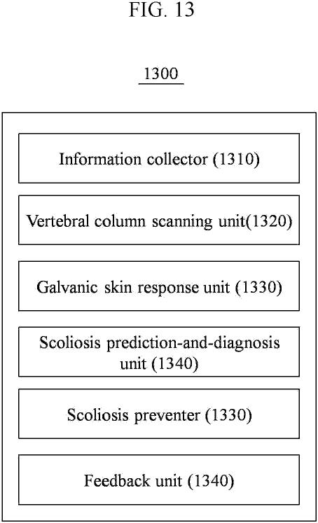

FIG. 12 is a diagram illustrating an example of an electronic device according to an example embodiment.

Referring to FIG. 12, an electronic device 1200 according to an example embodiment may include at least one of an input module 1210, an output module 1220, a memory 1230, and a processor 1240.

The input module 1210 may receive an instruction or data to be used for a component of the electronic device 1200 from an outside of the electronic device 1200. The input module 1210 may include at least one of an input device configured for a user to directly input an instruction or data to the electronic device 1200 and a communication device configured to receive an instruction or data through communication with an external electronic device in a wired or wireless manner. For example, the input device may include at least one of a microphone, a mouse, a keyboard, and a camera. For example, the communication device may include at least one of a wired communication device and a wireless communication device and the wireless communication device may include at least one of a near field communication device and a far field communication device.

The output module 1220 may provide information to the outside of the electronic device 1200. The output module 1220 may include at least one of an audio output device configured to auditorily output information, a display device configured to visually output information, and a communication device configured to transmit information through communication with the external electronic device in a wired or wireless manner. For example, the communication device may include at least one of a wired communication device and a wireless communication device and the wireless communication device may include at least one of a near field communication device and a far field communication device.

The memory 1230 may store data used by a component of the electronic device 1200. Data may include input data or output data related to a program or an instruction related thereto. For example, the memory 1230 may include at least one of a volatile memory and a nonvolatile memory.

The processor 1240 may control a component of the electronic device 1200 and may perform data processing or operation by executing the program of the memory 1230. Here, the processor 1240 may include a vertebral column scanning unit, a scoliosis prediction-and-diagnosis unit, and a scoliosis preventer, and may further include an information collector, a galvanic skin response unit, and a feedback unit. Through this, the processor 1240 may predict and diagnose scoliosis.

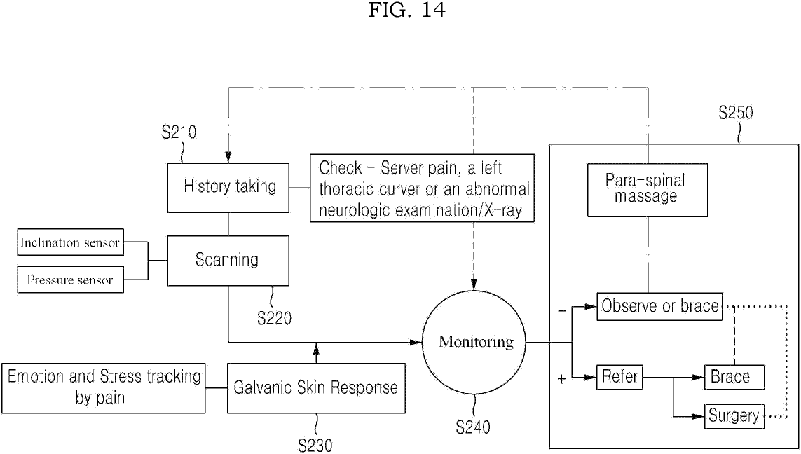

FIG. 13 is a diagram illustrating an example of an electronic device for predicting and diagnosing scoliosis according to an example embodiment.

Referring to FIG. 13, an electronic device 1300 for predicting and diagnosing scoliosis according to an example embodiment may include a vertebral column scanning unit 1320, a scoliosis prediction-and-diagnosis unit 1340, and a scoliosis preventer 1350, and may further include an information collector 1310, a galvanic skin response unit 1330, and a feedback unit 1360 depending on example embodiments. Here, the electronic device 1300 for predicting and diagnosing scoliosis may be included in the processor 1040 of FIG. 12 or may include the processor 1240.

Initially, the information collector 1310 may collect information on at least one of pain in the vertebral column, thoracic curve, neurological abnormal findings, and x-ray study.

For example, the information collector 1310 may receive information according to neurological examination history taking by inputting information of the subject to the input device through a manager, such as a doctor, or by receiving input of the information from the subject. Also, the information collector 1310 may collect severe pain in the vertebral column, thoracic curve, abnormal neurologic findings, or x-ray study history. This may be used to predict pain through a degree of curvature of the vertebral column measured or to monitor the degree of curvature of the vertebral column based on the collected information through the scoliosis prediction-and-diagnosis unit 1340.

The vertebral column scanning unit 1320 may measure the degree of curvature of the vertebral column through the pressure or the inclination of left and right sides of the vertebral column using the sensor. The vertebral column scanning unit 1320 may track a vertebral level or a peripheral nerve through vertebral column scanning using the sensor. Here, a device or a pushing rod that is a vertebral column scanning device may be used and the device or the pushing rod may be connected to a pressure sensor and/or an inclination sensor. As described above, the pressure may be measured or the inclination may be scanned from P to A (back to front) or A to P (front to back) by passing along the vertebral column using a round device or a rod-type object. Here, the degree of curvature of the vertebral column may be measured using an object in another shape in addition to the round device or the rod-type subject.

Meanwhile, the galvanic skin response unit 1330 may track an emotional or physiological change through galvanic skin response. Therefore, the scoliosis preventer 1350 may prevent scoliosis by massaging according to the tracked emotional or physiological change.

The scoliosis prediction-and-diagnosis unit 1340 may predict pain likely to occur in the vertebral column through the degree of curvature of the vertebral column or may compare before and after and monitor the degree of curvature of the vertebral column measured each time.

In detail, the scoliosis prediction-and-diagnosis unit 1340 may monitor the degree of curvature of the vertebral column according to a result of the vertebral column scanning unit 1320 and may continuously verify the vertebral level associated with the emotional or physiological phenomenon tracked through the galvanic skin response unit 1330 and may identify a pain area. Here, for correlation between the vertebral level associated with the emotional or physiological phenomenon and the pain area, the information collector 1310 may collect information such as a type of pain or emotion according to the pain area of the spinal column.

The scoliosis preventer 1350 may prevent scoliosis by massaging the pain area or a portion with a relatively great difference in the pressure or the inclination through an increase-and-decrease prediction and diagnosis of a curve that represents the degree of curvature of the vertebral column according to a monitoring result.

The feedback unit 1360 may reselect and operate a previous process through feedback after preventing the scoliosis by massaging the pain area or the portion with the relatively great difference in the pressure or the inclination. That is, the feedback unit 1360 may measure the degree of curvature of the vertebral column again and may compare before and after and monitor the degree of curvature of the vertebral column.

FIG. 14 illustrates an example of an operating method of an electronic device for predicting and diagnosing scoliosis according to an example embodiment.

Referring to FIG. 14, the operating method of the electronic device for predicting and diagnosing scoliosis according to an example embodiment may include operation S220 of measuring a degree of curvature of vertebral column through pressure or inclination of left and right sides of the vertebral column using a sensor, operation S240 of predicting pain likely to occur in the vertebral column through the degree of curvature of the vertebral column or comparing before and after and monitoring the degree of curvature of the vertebral column measured each time, and operation S250 of preventing scoliosis by massaging a pain area or a portion with a relatively great difference in the pressure or the inclination through an increase-and-decrease prediction and diagnosis of a curve that represents the degree of curvature of the vertebral column according to a monitoring result.

Here, the method may further include operation S210 of collecting information on at least one of pain in the vertebral column, thoracic curve, neurological abnormal findings, and x-ray study prior to verifying the degree of curvature of the vertebral column.

Also, the method may further include operation S230 of tracking the emotional or physiological change through the galvanic skin response.

Also, the method may further include operation of reselecting and operating a previous process through feedback after preventing the scoliosis by massaging the pain area or the portion with the relatively great difference in the pressure or the inclination.

Hereinafter, each operation of the operating method of the electronic device for predicting and diagnosing scoliosis according to an example embodiment will be further described.

An operating method of an electronic device predicting and diagnosing scoliosis according to an example embodiment will be described in detail by using, as an example, an electronic device for predicting and diagnosing scoliosis described with reference to FIG. 13. The electronic device 1300 for predicting and diagnosing scoliosis according to an example embodiment may include the vertebral column scanning unit 1320, the scoliosis prediction-and-diagnosis unit 1340, and the scoliosis preventer 1350, and may further include the information collector 1310, the galvanic skin response unit 1330, and the feedback unit 1360 depending on example embodiments.

In operation S210, the information collector 1310 may collect information on at least one of pain in the vertebral column, thoracic curve, neurological abnormal findings, and x-ray study. This may be used to predict pain through a degree of curvature of the vertebral column measured or to monitor the degree of curvature of the vertebral column based on the collected information through the scoliosis prediction-and-diagnosis unit 1340.

In operation S220, the vertebral column scanning unit 1320 may measure the degree of curvature of the vertebral column through the pressure or the inclination of left and right sides of the vertebral column using the sensor.

Here, the vertebral column scanning unit 1320 may scan the vertebral column using a device or may scan the vertebral column using a pushing rod.

For example, the vertebral column scanning unit 1320 may scan the vertebral column using the device that moves along the vertebral column and may measure the degree of curvature of the vertebral column through the pressure or inclination of the left and the right sides of the vertebral column using a sensor connected to the device.

As another example, the vertebral column scanning unit 1320 may scan the vertebral column using the pushing rod that is pressed along the vertebral column and may measure the degree of curvature of the vertebral column through the pressure or the inclination of the left and right sides of the vertebral column using a sensor connected to the pushing rod.

The vertebral column scanning unit 1320 may use the sensor to measure the degree of curvature of the vertebral column through pressure or inclination of left and right sides of the vertebral column. For example, a pressure sensor and an inclination sensor may be used.

For example, the vertebral column scanning unit 1320 may measure the pressure of the left and right sides of the vertebral column through at least one pressure sensor that is connected to the device or the pushing rod and, through this, may measure the degree of curvature of the vertebral column. For example, two pressure sensors may be used on the left and right sides of the vertebral column.

As another example, the vertebral column scanning unit 1320 may measure the inclination of the vertebral column through the inclination sensor that is connected to the device or the pushing rod and, through this, may measure the degree of curvature of the vertebral column.

As another example, the vertebral column scanning unit 1320 may measure the pressure and the inclination of the left and right sides of the vertebral column using all of the pressure sensor and the inclination sensor that are connected to the device or the pushing rod and through this, may measure the degree of curvature of the vertebral column. Here, referring to FIG. 1, a plurality of pressure sensors may be provided to the left and right sides of the vertebral column and the inclination sensor may be provided at the center of the plurality of pressure sensors.

In operation S230, the galvanic skin response unit 1330 may track the emotional or physiological change through the galvanic skin response. Therefore, the scoliosis preventer 1350 may prevent the scoliosis by massaging according to the tracked emotional or physiological change. That is, the galvanic skin response unit 1330 may track emotion and stress caused by pain and then may apply the same for massaging for prevention while monitoring severe pain likely to occur in the vertebral column.

In operation S240, the scoliosis prediction-and-diagnosis unit 1340 may predict pain likely to occur in the vertebral column through the degree of curvature of the vertebral column or may compare before and after and monitor the degree of curvature of the vertebral column measured each time.

In operation S250, the scoliosis preventer 1350 may maintain muscle relaxation and joint range of motion and prevent scoliosis accordingly by intensively massaging the pain area or the portion with the relatively great difference in the pressure or the inclination through an increase-and-decrease prediction and diagnosis of a curve that represents the degree of curvature of the vertebral column according to a monitoring result. For example, the scoliosis preventer 1350 may intensively massage the pain area or the portion with the relatively great difference in the pressure or the inclination by referring to information of Table 1.

Table 1 represents treatment and referral guidelines for patients of scoliosis.

TABLE 1

| CURVE(DEGREES) | RISSER GRADE | X-RAY/REFER | TREATMENT |
|---|---|---|---|
| 10 to 19 | 0 to 1 | Every 6 months/no | |
| 10 to 19 | 2 to 4 | Every 6 months/no | Observe |
| 20 to 29 degrees | 0 to 1 | Every 6 months/yes | Brace after 25 |
| 20 to 29 | 2 to 4 | Every 6 months/yes | Observe or brace* |
| 20 to 40 | 0 to 1 | Refer | Brace |
| 20 to 40 | 2 to 4 | Refer | Brace |
| >40 | 0 to 4 | Refer | Surgery |

The feedback unit 1360 may reselect and operate a previous process through feedback after preventing the scoliosis by massaging the pain area or the portion with the relatively great difference in the pressure or the inclination. That is, the feedback unit 1360 may measure the degree of curvature of the vertebral column again and may compare before and after and monitor the degree of curvature of the vertebral column.

As described above, according to some example embodiments, it is possible to measure or diagnose scoliosis by measuring a degree of curvature of vertebral column through pressure or inclination of left and right sides of the vertebral column and to prevent the scoliosis by massaging a pain area or a portion with a relatively great difference in the pressure or the inclination.

The apparatuses described herein may be implemented using hardware components, software components, and/or a combination thereof. For example, the apparatuses and the components described herein may be implemented using one or more general-purpose or special purpose computers, such as, for example, a processor, a controller, an arithmetic logic unit (ALU), a digital signal processor, a microcomputer, a field programmable array (FPA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The software may include a computer program, a piece of code, an instruction, or some combinations thereof, for independently or collectively instructing or configuring the processing device to operate as desired. Software and/or data may be embodied in any type of machine, component, physical equipment, virtual equipment, or a computer storage medium or device, to be interpreted by the processing device or to provide an instruction or data to the processing device. The software also may be distributed over network coupled computer systems so that the software is stored and executed in a distributed fashion. The software and data may be stored by one or more computer readable storage media.

The methods according to the above-described example embodiments may be configured in a form of program instructions performed through various computer devices and recorded in computer-readable media. The media may also include, alone or in combination with the program instructions, data files, data structures, and the like. The program instructions recorded in the media may be specially designed and configured for the example embodiments or may be known to those skilled in the computer software art and thereby available. Examples of the media include magnetic media such as hard disks, floppy disks, and magnetic tapes; optical media such as CD-ROM and DVDs; magneto-optical media such as floptical disks; and hardware devices that are specially configured to store program instructions, such as read-only memory (ROM), random access memory (RAM), flash memory, and the like. Examples of program instructions include both machine code, such as produced by a compiler, and files containing higher level code that may be executed by the computer using an interpreter.

While the example embodiments are described with reference to specific example embodiments and drawings, it will be apparent to one of ordinary skill in the art that various alterations and modifications in form and details may be made in these example embodiments without departing from the spirit and scope of the claims and their equivalents. For example, suitable results may be achieved if the described techniques are performed in a different order, and/or if components in a described system, architecture, device, or circuit are combined in a different manner, or replaced or supplemented by other components or their equivalents.

Therefore, other implementations, other example embodiments, and equivalents of the claims are to be construed as being included in the claims.

What is claimed is:

1. An operating method of an electronic device for predicting and diagnosing scoliosis, the method comprising:
measuring a degree of curvature of vertebral column through pressure or inclination of left and right sides of the vertebral column using a sensor;
predicting pain likely to occur in the vertebral column through the degree of curvature of the vertebral column or comparing before and after and monitoring the degree of curvature of the vertebral column measured each time; and
preventing scoliosis by massaging a pain area or a portion with a relatively great difference in the pressure or the inclination through an increase-and-decrease prediction and diagnosis of a curve that represents the degree of curvature of the vertebral column according to a monitoring result.

2. The method of claim 1, further comprising:
collecting information on at least one of pain in the vertebral column, thoracic curve, neurological abnormal findings, and x-ray study prior to verifying the degree of curvature of the vertebral column,
wherein the pain is predicted or the degree of curvature of the vertebral column is monitored through the degree of curvature of the vertebral column that is measured based on the collected information.

3. The method of claim 1, further comprising:
tracking an emotional or physiological change through a galvanic skin response,
wherein the scoliosis is prevented by massaging according to the tracked emotional or physiological change.

4. The method of claim 1, further comprising:
reselecting and operating a previous process through feedback after preventing the scoliosis by massaging the pain area or the portion with the relatively great difference in the pressure or the inclination,
wherein the reselecting and the operating the previous process through the feedback comprises measuring the degree of curvature of the vertebral column again and comparing before and after and monitoring the degree of curvature of the vertebral column.

5. The method of claim 1, wherein the measuring the degree of curvature of the vertebral column comprises scanning the vertebral column using a device that moves along the vertebral column and measuring the degree of curvature of the vertebral column through the pressure or the inclination of the left and the right sides of the vertebral column using a sensor connected to the device.

6. The method of claim 1, wherein the measuring the degree of curvature of the vertebral column comprises scanning the vertebral column using a pushing rod that is pressed along the vertebral column and measuring the degree of curvature of the vertebral column through the pressure or the inclination of the left and right sides of the vertebral column using a sensor connected to the pushing rod.

7. An electronic device for predicting and diagnosing scoliosis, the electronic device comprising:
a vertebral column scanning unit configured to measure a degree of curvature of vertebral column through pressure or inclination of left and right sides of the vertebral column using a sensor;
a scoliosis prediction-and-diagnosis unit configured to predict pain likely to occur in the vertebral column through the degree of curvature of the vertebral column or to compare before and after and monitor the degree of curvature of the vertebral column measured each time; and
a scoliosis preventer configured to prevent scoliosis by massaging a pain area or a portion with a relatively great difference in the pressure or the inclination through an increase-and-decrease prediction and diagnosis of a curve that represents the degree of curvature of the vertebral column according to a monitoring result.

8. The electronic device of claim 7, further comprising:
an information collector configured to collect information on at least one of pain in the vertebral column, thoracic curve, neurological abnormal findings, and x-ray study,
wherein the scoliosis prediction-and-diagnosis unit is configured to predict the pain or monitor the degree of curvature of the vertebral column through the degree of curvature of the vertebral column that is measured based on the collected information.

9. The electronic device of claim 7, further comprising:
a galvanic skin response unit configured to track an emotional or physiological change through a galvanic skin response,
wherein the scoliosis preventer is configured to prevent the scoliosis by massaging according to the tracked emotional or physiological change.

10. The electronic device of claim 7, further comprising:
a feedback unit configured to reselect and operate a previous process through feedback after preventing the scoliosis by massaging the pain area or the portion with the relatively great difference in the pressure or the inclination,
wherein the feedback unit is configured to measure the degree of curvature of the vertebral column again and to compare before and after and monitor the degree of curvature of the vertebral column.
